(19) United States
(12) Patent Application Publication
(10) Pub. No.: US 2007/0161797 A1
(43) Pub. Date: Jul. 12, 2007

(54) Process for the manufacture of 2,3-dichloropyridine

(76) Inventor: Rafael Shapiro, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/583,635

(22) PCT Filed: Jan. 21, 2005
(86) PCT No.: PCT/US05/02462

§ 371 Date: Jun. 20, 2006

Related U.S. Application Data
(60) Provisional application No. 60/539,068, filed on Jan. 23, 2004.

(51) Int. Cl. C07D 213/61

(52) U.S. Cl. 546/345

(57) ABSTRACT

A method for preparing 2,3-dichloropyridine is disclosed in which 3-amino-2-chloropyridine is contacted with an alkali metal nitrite in the presence of aqueous hydrochloric acid to form a diazonium salt; and the diazonium salt is subsequently decomposed in the presence of copper catalyst wherein at least about 50% of the copper is the copper(II) oxidation state.

### BACKGROUND OF THE INVENTION

A need exists for efficient and practical processes for the manufacture of 2,3-dichloropyridine. 2,3-Dichloropyridine is an important raw material for the preparation of crop protection agents, pharmaceuticals and other fine chemicals.

H. J. den Hertog, et al., Recl. Trav. Chim. Pays-Bas, 1950, 69, 673, report the preparation of 2,3-dichloropyridine from 3-amino-2-chloropyridine by the Gatterman reaction, in which copper powder was used as a catalyst. However, the usefulness of the reported method is severely limited with respect to low yield cited (about 45%) and limited scale (about 1 g).

### SUMMARY OF THE INVENTION

This invention relates to a method of preparing 2,3-dichloropyridine 1, 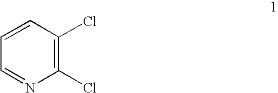
comprising the steps of:
(1) contacting 3-amino-2-chloropyridine 2 or a solution comprising 3-amino-2-chloropyridine 2 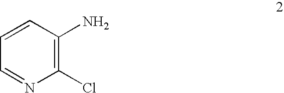
with hydrochloric acid to form a 3-amino-2-chloropyridine hydrochloric acid salt;
(2) contacting the 3-amino-2-chloropyridine hydrochloric acid salt with a nitrite salt to form a corresponding diazonium chloride salt; and
(3) contacting the corresponding diazonium chloride salt with hydrochloric acid in the presence of a copper catalyst wherein at least about 50% of the copper is the copper (II) oxidation state, optionally in the presence of an organic solvent, to form 2,3-dichloropyridine 1.

This invention also relates to the above method of preparing 2,3-dichloropyridine 1, wherein the 3-amino-2-chloropyridine 2 or the solution comprising the 3-amino-2-chloropyridine 2 is prepared by a method comprising the steps of:
(a) contacting 3-aminopyridine 3 or a solution comprising 3-aminopyridine 3 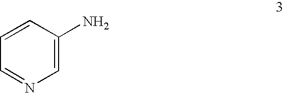
with hydrochloric acid to form a 3-aminopyridine hydrochloric acid salt;
(b) contacting the 3-aminopyridine hydrochloric acid salt with a chlorinating agent to form the solution comprising the 3-amino-2-chloropyridine 2; and
(c) optionally isolating the 3-amino-2-chloropyridine 2 from the solution of step (b).

This invention also relates to the above methods of preparing 2,3-dichloropyridine 1 wherein the 3-aminopyridine 3 or the solution comprising the 3-aminopyridine 3 is prepared by a method comprising the steps of:
(i) contacting nicotinamide 4 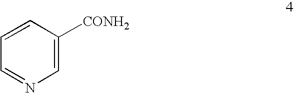
with a strong base and a halogenating agent to form a mixture comprising an N-halonicotinamide salt;
(ii) contacting the N-halonicotinamide salt mixture formed in step (i) with heated water to form an aqueous mixture and maintaining the aqueous mixture at a temperature ranging from about 65 to about 100° C. to form the solution comprising the 3-aminopyridine 3;
(iii) isolating the 3-aminopyridine 3 from the solution of step (ii) if the halogenating agent is other than a chlorinating agent; and
(iv) optionally isolating the 3-aminopyridine 3 from the solution of step (ii) if the halogenating agent is a chlorinating agent.

### DETAILED DESCRIPTION OF THE INVENTION

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

Embodiments of the present invention include:
Embodiment A. A method (Method A) of preparing 2,3-dichloropyridine 1, 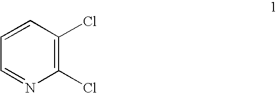
comprising the steps of:

(1) contacting a solution comprising 3-amino-2-chloropyridine 2 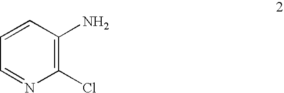
with a first aqueous solution comprising hydrochloric acid to form 3-amino-2-chloropyridine hydrochloric acid salt;
(2) contacting the 3-amino-2-chloropyridine hydrochloric acid salt with an aqueous solution comprising a nitrite salt to form a diazonium salt; and
(3) contacting the diazonium salt with an aqueous solution comprising a Cu(II) salt in the presence of a second aqueous solution comprising hydrochloric acid, optionally in the presence of an organic solvent, to form 2,3-dichloropyridine 1.
Embodiment 1. A method of Embodiment A wherein the nitrite salt is sodium nitrite.
Embodiment 2. A method of Embodiment A wherein the Cu(II) salt is copper(II) chloride or copper (II) oxide.
Embodiment 3. A method of Embodiment A wherein
   the nominal mole ratio of the nitrite salt to 3-amino-2-chloropyridine is about 0.95 to about 2.0;
   the nominal mole ratio of the Cu(II) salt to 3-amino-2-chloropyridine is about 0.05 to about 2.0;
   the nominal mole ratio of the hydrochloric acid in the first aqueous solution to 3-amino-2-chloropyridine is about 3 to about 10; and
   the nominal mole ratio of the hydrochloric acid in the second aqueous solution to 3-amino-2-chloropyridine is about 0 to about 10.
Embodiment 4. The method of Embodiment 3 wherein
   the nominal mole ratio of the nitrite salt to 3-amino-2-chloropyridine is about 0.95 to about 1.1;
   the nominal mole ratio of the Cu(II) salt to 3-amino-2-chloropyridine is about 0.2 to about 0.6;
   the nominal mole ratio of the hydrochloric acid in the first aqueous solution to 3-amino-2-chloropyridine is about 3 to about 6; and
   the nominal mole ratio of the hydrochloric acid in the second aqueous solution to 3 amino-2-chloropyridine is about 1 to about 5.
Embodiment 5. A method of Embodiment A wherein
   steps (1) and (2) are conducted at a temperature ranging from about −15 to about 20° C.; and
   step (3) is conducted at a temperature ranging from about 30 to about 90° C.
Embodiment 6. The method of Embodiment 5 wherein
   the temperature of steps (1) and (2) range from about −10 to about 10° C.; and
   the temperature of step (3) ranges from about 50 to about 80° C.
Embodiment B. A method (Method B) of preparing 2,3-dichloropyridine 1, comprising the steps of:
(a) contacting a solution comprising 3-aminopyridine 3 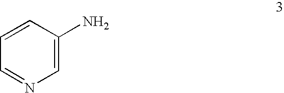
with aqueous hydrochloric acid and a chlorinating agent to form a mixture;
(b) isolating a solution comprising 3-amino-2-chloropyridine hydrochloric acid salt from the mixture; and
(c) using the solution comprising 3-amino-2-chloropyridine hydrochloric acid salt in the method of Embodiment A described above for the preparation of 2,3-dichloropyridine.
Embodiment a. A method of Embodiment B wherein the chlorinating agent is chlorine, an alkali metal hypochlorite or a mixture of hydrochloric acid and hydrogen peroxide.
Embodiment b. The method of Embodiment a wherein the chlorinating agent is chlorine or a mixture of hydrogen peroxide and hydrochloric acid.
Embodiment c. A method of Embodiment B wherein
   the nominal mole ratio of hydrochloric acid to 3-aminopyridine is about 3 to about 20; and
   the nominal mole ratio of the chlorinating agent to 3-aminopyridine is about 0.6 to about 1.5.
Embodiment d. The method of Embodiment c wherein
   the nominal mole ratio of hydrochloric acid to 3-aminopyridine is about 5 to about 15; and
   the nominal mole ratio of the chlorinating agent to 3-aminopyridine is about 0.8 to about 1.2.
Embodiment e. A method of Embodiment B wherein step (a) is conducted at a temperature ranging from about 0 to about 60° C.
Embodiment f. The method of Embodiment e wherein the temperature of step (a) ranges from about 10 to about 35° C.
Embodiment C. A method (Method C) of preparing 2,3-dichloropyridine 1 comprising the steps of:
   (i) contacting nicotinamide 4 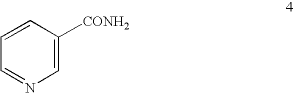
with a strong base and a halogenating agent in an aqueous solution at a temperature ranging from about −5 to about 20° C. to form a mixture comprising an N-halonicotinamide salt;
   (ii) contacting the N-halonicotinamide salt mixture generated in step (i) with water and maintaining a resulting aqueous mixture at a temperature ranging from about 65 to about 100° C.;
   (iii) isolating a solution comprising 3-aminopyridine hydrochloric acid salt from the aqueous mixture of step (ii); and
   (iv) using the solution comprising 3-aminopyridine hydrochloric acid salt in Method B described above for the preparation of 2,3-dichloropyridine.
Embodiment i. A method of Embodiment C wherein the strong base is an alkali metal hydroxide.
Embodiment ii. The method of Embodiment i wherein the alkali metal hydroxide is sodium hydroxide.
Embodiment iii. A method of Embodiment C wherein the halogenating agent is chlorine, bromine, or sodium hypochlorite.
Embodiment iv. A method of Embodiment C wherein
   the nominal mole ratio of the strong base to nicotinamide is about 1 to about 5; and
   the nominal mole ratio of the halogenating agent to nicotinamide is from about 0.8 to about 2.0.
Embodiment v. The method of Embodiment iv wherein
   the nominal mole ratio of the strong base to nicotinamide is about 2 to about 4 when the halogenating agent is chlorine or bromine;
   the nominal mole ratio of the strong base to nicotinamide is about 1 to about 2 when the halogenating agent is sodium hypochlorite; and
   the nominal mole ratio of the halogenating agent to nicotinamide is about 0.9 to about 1.1.
Embodiment vi. A method of Embodiment vi wherein
   the temperature of step (i) ranges from about 0 to about 10° C.; and
   the temperature of step (ii) ranges from about 70 to about 95° C.
Embodiment B′. A method (Method B′) of preparing 2,3-dichloropyridine 1, comprising the steps of:
(a′) contacting a solution comprising 3-aminopyridine 3 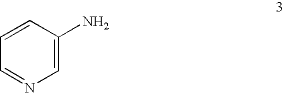
with aqueous hydrochloric acid and a chlorinating agent to form a solution comprising 3-amino-2-chloropyridine hydrochloric acid salt;
(b′) optionally isolating 3-amino-2-chloropyridine 2 from the solution of step (a′); and
(c′) using the solution of step (a′) or the 3-amino-2-chloropyridine 2 of step (b′) in Embodiment A for the preparation of 2,3 -dichloropyridine 1.

The Embodiments a-f above to further describe Embodiment B (Method B) are also Embodiments of Embodiment B′ (Method B′).
Embodiment C′. A method (Method C′) of preparing 2,3-dichloropyridine 1 comprising the steps of:
   (i′) contacting nicotinamide 4 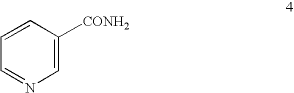
   with a strong base and a halogenating agent in an aqueous solution at a temperature ranging from about −5 to about 20° C. to form a mixture comprising an N-halonicotinamide salt;
   (ii′) contacting the N-halonicotinamide salt mixture generated in step (i′) with heated water to form an aqueous mixture and maintaining the aqueous mixture at a temperature ranging from about 65 to about 100° C. to form a solution comprising 3-aminopyridine 3;
   (iii′) optionally isolating the 3-aminopyridine 3 from the aqueous mixture of step (ii′); and
   (iv′) using the solution of step (ii′) if the halogenating agent is a chlorinating agent or the 3-aminopyridine 3 of step (iii′) in Embodiment B′ for the preparation of 3-amino-2-chloropyridine 2.

The Embodiments i-vi above to further describe Embodiment C (Method C) are also Embodiments of Embodiment C′ (Method C′).

Embodiment AA. A method of preparing 2,3-dichloropyridine 1 as set forth in the Summary of the Invention wherein the nitrite salt is sodium nitrite.

Embodiment BB. The method of preparing 2,3-dichloropyridine 1 as set forth in the Summary of the Invention wherein at least about 75% of the copper is the copper(II) oxidation state.

Embodiment CC. The method of Embodiment BB wherein at least about 90% of the copper is the copper(II) oxidation state.

Embodiment DD. The method of Embodiment CC wherein at least about 95% of the copper is the copper(II) oxidation state.

Embodiment EE. The method of Embodiment DD wherein at least about 99% of the copper is the copper(II) oxidation state.

Embodiment FF. The method of Embodiment EE wherein 100% of the copper is the copper(II) oxidation state.

Embodiment GG. The method of preparing 2,3-dichloropyridine 1 as set forth in the Summary of the Invention wherein the copper catalyst comprises copper(II) chloride or copper(II) oxide.

Embodiment HH. The method of Embodiment GG wherein the nominal mole ratio of the nitrite salt to the 3-amino-2-chloropyridine 2 is about 0.95 to about 2.0; the nominal mole ratio of the copper(II) chloride or the copper(II) oxide to the 3-amino-2-chloropyridine 2 is about 0.05 to about 2.0 when 100% of the copper is copper(II) chloride or copper(II) oxide; the nominal mole ratio of hydrochloric acid to the 3-amino-2-chloropyridine 2 in step (1) is about 3 to about 10; and the nominal mole ratio of hydrochloric acid to the 3-amino-2-chloropyridine 2 in step (3) is about 0 to about 10.

Embodiment II. The method of Embodiment HH wherein the nominal mole ratio of the nitrite salt to the 3-amino-2-chloropyridine 2 is about 0.95 to about 1.1; the nominal mole ratio of the copper in the copper catalyst to the 3-amino-2-chloropyridine 2 is about 0.2 to about 0.6; the nominal mole ratio of the hydrochloric acid to 3-amino-2-chloropyridine 2 in step (1) is about 3 to about 6; and the nominal mole ratio of the hydrochloric acid to 3-amino-2-chloropyridine 2 in step (3) is about 1 to about 5.

Embodiment JJ. The method of preparing 2,3-dichloropyridine 1 as set forth in the Summary of the Invention wherein steps (1) and (2) are conducted at a temperature ranging from about −15 to about 20° C.; and step (3) is conducted at a temperature ranging from about 30 to about 90° C.

Embodiment KK: The method of Embodiment JJ wherein steps (1) and (2) are conducted at a temperature ranging from about −10 to about 10° C.; and step (3) is conducted at a temperature ranging from about 50 to about 80° C.

Embodiment LL: The method of preparing 2,3-dichloropyridine 1 as set forth in the Summary of the Invention wherein the chlorinating agent is chlorine, an alkali metal hypochlorite or a mixture of hydrochloric acid and hydrogen peroxide.

Embodiment MM: The method of Embodiment LL wherein the chlorinating agent is chlorine or a mixture of hydrochloric acid and hydrogen peroxide.

Embodiment NN: The method of preparing 2,3-dichloropyridine 1 as set forth in the Summary of the Invention wherein the nominal mole ratio of hydrochloric acid to 3-aminopyridine 3 in step (a) is about 3 to about 20; and the nominal mole ratio of the chlorinating agent to the 3-aminopyridine 3 in step (a) is about 0.6 to about 1.5.

Embodiment OO: The method of Embodiment NN wherein the nominal mole ratio of hydrochloric acid to the 3-aminopyridine 3 in step (a) is about 5 to about 15; and the nominal mole ratio of the chlorinating agent to the 3-aminopyridine 3 in step (a) is about 0.8 to about 1.2.

Embodiment PP: The method of preparing 2,3-dichloropyridine 1 as set forth in the Summary of the Invention wherein steps (a) and (b) are conducted at a temperature ranging from about 0 to about 60° C.

Embodiment QQ: The method of Embodiment PP wherein steps (a) and (b) are conducted at a temperature ranging from about 10 to about 35° C.

Embodiment RR: The method of preparing 2,3-dichloropyridine 1 as set forth in the Summary of the Invention wherein the strong base is an alkali metal hydroxide.

Embodiment SS: The method of Embodiment RR wherein the alkali metal hydroxide is sodium hydroxide.

Embodiment TT: The method of preparing 2,3-dichloropyridine 1 as set forth in the Summary of the Invention wherein the halogenating agent is chlorine, bromine, or sodium hypochlorite.

Embodiment UU: The method of preparing 2,3-dichloropyridine 1 as set forth in the Summary of the Invention wherein the nominal mole ratio of the strong base to nicotinamide 4 is about 1 to about 5; and the nominal mole ratio of the halogenating agent to nicotinamide 4 is about 0.8 to about 2.0.

Embodiment VV: The method of Embodiment UU wherein the nominal mole ratio of the strong base to nicotinamide 4 is about 2 to about 4 when the halogenating agent is chlorine or bromine; the nominal mole ratio of the strong base to nicotinamide 4 is about 1 to about 2 when the halogenating agent is sodium hypochlorite; and the nominal mole ratio of halogenating to nicotinamide 4 is about 0.9 to about 1.1.

Embodiment WW: The method of preparing 2,3-dichloropyridine 1 as set forth in the Summary of the Invention wherein step (i) is conducted at a temperature ranging from about −5 to about 20° C.

Embodiment XX: The method Embodiment WW wherein step (i) is conducted at a temperature ranging from about 0 to about 10° C.; and step (ii) is conducted at a temperature ranging from about 70 to about 95° C.

According to the present invention, e.g., Method A, as shown in Scheme 1, 2,3-dichlioropyridine 1 is prepared by diazotization of 2-chloro-3-aminopyridine 2 followed by decomposition of the diazonium chloride salt in the presence of a Cu(II) salt, i.e. in the presence of a copper catalyst wherein at least about 50% of the copper is the copper(II) oxidation state. 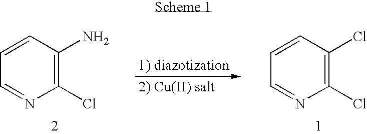
The diazonium chloride salt can be prepared by reaction of 3-amino-2-chloropyridine 2 with nitrous acid in an aqueous solution at a suitable temperature. The nitrous acid can be generated in situ from a nitrite salt and hydrochloric acid. Various nitrite salts can be used, such as sodium nitrite, potassium nitrite, calcium nitrite, or any alkali or alkali earth nitrite. A suitable nitrite salt is sodium nitrite for the reasons of cost and availability. For references on how to prepare diazonium salt see H. Zollinger, Azo and Diazo Chemistry, Wiley-Interscience, New York, 1961; S Patai, The Chemistry of Diazonium and Diazo Groups, Wiley, New York, 1978, Chapters 8, 11 and 14; and H. Saunders and R. L. M. Allen, Aromatic Diazo Compounds, Third Edition, Edward Arnold, London, 1985. In one embodiment of the process of the present invention, a solution comprising 3-amino-2-chloropyridine 2 is contacted with a first aqueous solution comprising hydrochloric acid to form 3-amino-2-chloropyridine hydrochloric acid salt. The 3-amino-2-chloropyridine hydrochloric acid salt is then contacted with an aqueous solution comprising a nitrite salt to form a diazonium chloride salt. Diazotization of the 3-amino-2-chloropyridine hydrochloric acid salt is suitably accomplished by adding aqueous sodium nitrite to a mixture of the 3-amino-2-chloropyridine 2 in about 10% to about 37% aqueous hydrochloric acid. Additional embodiments for these steps of the present method, for example but not limitation Method A, are described above.

The diazonium chloride salt is decomposed in the presence of hydrochloric acid and a copper catalyst wherein at least about 50% of the copper is the copper (II) oxidation state to form 2,3-dichloropyridine 1. In additional embodiments, at least about 75%, at least about 90%, at least about 95%, at least about 99%, or 100% of the copper is the copper (II) oxidation state. The copper catalyst can comprise, for example but not limitation, copper(II) acetate, copper(II) nitrate, copper(II) sulfate, copper(II) oxide (CuO), or copper(II) chloride (CuCl₂). In one embodiment the copper catalyst comprises copper(II) oxide (CuO), copper(II) chloride (CuCl₂), or copper(II) chloride generated in situ from CuO and hydrochloric acid (HCl). In other embodiments at least 75% of the copper is copper(II) chloride; at least 90% of the copper is copper(II) chloride; at least 99% of the copper is copper(II) chloride; at least 99% of the copper is copper(II) chloride; 100% of the copper is copper(II) chloride; at least 75% of the copper is copper(II) oxide; at least 90% of the copper is copper(II) oxide; at least 95% of the copper is copper(II) oxide; at least 99% of the copper is copper(II) oxide; and 100% of the copper is copper(II) oxide.

The decomposition can be conducted in an aqueous solution, i.e., a one-phase system, comprising about 0 to about 10, about 1 to about 5, mole equivalent (relative to 3-amino-2-chloropyridine 2) of about 10% to about 37% aqueous HCl, and about 0.05 to about 2, about 0.2 to about 0.6 mol equivalent (relative to 3-amino-2-chloropyridine 2) of copper catalyst at a temperature ranging from about 30 to about 90° C. In one embodiment the decomposition temperature is about 50 to about 80° C. The product, 2,3-dichloro-pyridine 1, in the one-phase system, can be isolated by allowing the reaction mixture cooled to ambient temperature, optionally addition of a base to neutralize the reaction mixture, followed by filtration.

The decomposition can also be conducted in a two-phase system, comprising a suitable organic solvent and the aqueous solution of the one-phase system. The suitable organic solvent for the two-phase system can be, for example but not limitation, tetrahydrofuran, cyclohexane, ethyl acetate, n-chlorobutane, toluene, or benzene. The volume ratio of the organic phase and aqueous phase in the two-phase system can range from about 1:10 to about 10:1. The product, 2,3-dichloropyridine 1, in the two-phase system, can be isolated by dilution of the reaction mass with water or aqueous base, phase-separation, and concentration of the organic phase to dryness. The product of 2,3-dichloropyridine 1 can also be isolated from the organic phase from the phase-separation by crystallization. The crystallization can be achieved by partial concentration of the organic solution, and optional addition of an “antisolvent” such as heptane or water. By “antisolvent” is meant a liquid diluent which when added to a solution of the desired product reduces the solubility of the product in the resulting mixture. Thus, if the solvent is a polar solvent such as an amide or a lower alcohol, such as DMF or ethanol, water could be a suitable antisolvent. On the other hand, if the solvent is a moderately nonpolar solvent, such as ethyl acetate or dichloromethane, an appropriate antisolvent could be a very nonpolar or hydrocarbon solvent, such as cyclohexane or heptane. The isolated yield of 2,3-dichloro-pyridine 1 (ca. 98% purity) can be about 90-95% starting from pure 3-amino-2-chloropyridine 2. The aqueous phase from the phase-separation can be recycled directly into a subsequent decomposition batch, with optionally partial concentration, for the reuse of Cu(II) salt catalyst and excess hydrochloric acid.

According to this invention as shown in Scheme 2, e.g., Method B or Method B′, 2,3-dichioropyridine 1 can be prepared by chlorination of 3-aminopyridine 3 followed by diazotization of the resulting 2-chloro-3-aminopyridine 2 intermediate and decomposition of the diazonium chloride salt as described above, e.g., in Method A.

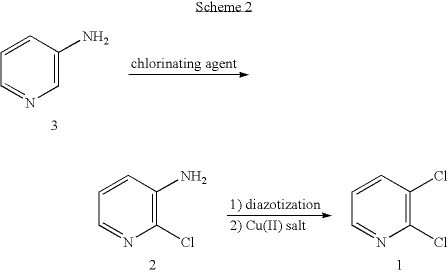

In one embodiment of the process of the present invention, a solution comprising 3-aminopyridine 3 is contacted with aqueous hydrochloric acid and a chlorinating agent to form a mixture. Chlorination of 3-aminopyridine 3 can be achieved by various suitable chlorinating agents, such as chlorine, alkali metal (such as lithium, sodium or potassium) hypochlorite, or a mixture of hydrochloric acid and hydrogen peroxide. Embodiments of chlorinating agents are also described above. 3-Amino-2-chloropyridine 2 is known to be prepared from 3-aminopyridine 3 by reacting the latter with hydrochloric acid and hydrogen peroxide at a temperature of 70-80° C. (O. von Schickh, A. Binz, and A. Schultz, Chem. Ber., 1936, 69, 2593). However, this method easily provides over-chlorinated products (e.g. 3-amino-2,6-dichlorpyridine) because of the relatively high reaction temperature. This method was optimized by Yuan et al. (Zhongguo Yiyao Gongye Zazhi, 2000, 31, 420), to lower the reaction temperature to 20-30° C. and to reduce the amount of over-chlorinated product to 8 wt % by using 1 mol equivalent of 15 wt % hydrogen peroxide and concentrated aqueous HCl (ca. 37 wt %).

3-Amino-2-chloropyridine 2 is also known to be prepared from 3-aminopyridine 3 by transition metal catalyzed chlorination of 3-aminopyridine 3 (Blank, et al., U.S. Pat. No. 3,838,136). This method, while providing better yields on production scale than von Schickh's method described above, has the limitations that a hazardous material (chlorine) is required, the product is isolated as a solid in relatively impure form (ca. 87 wt %), and the metal catalysts are not easily recyclable and thus constitute potential waste-disposal issues. Purification of 3-amino-2-chloropyridine 2, prepared by the method of Blank et al., from the by-product, 3-amino-2,6-dichloropyridine, was described by K. Ieno in JP 09227522.

In one embodiment of the present invention, a more selective chlorination method is used to produce higher quality 3-amino-2-chloropyridine 2 from 3-aminopyridine 3 by using a high strength hydrogen peroxide (about 20 to about 50 wt %), concentrated HCl, and a low temperature (about 10 to about 35° C.). This selective chlorination method can minimize over-chlorinated products (primarily 3-amino-2,6-dichloropyridine), even at a high conversion percentage of 3-aminopyridine 3. Furthermore, a modification of the Ieno's method allows for easy purification of 3-amino-2-chloropyridine 2 and continuation of the crude 3-amino-2-chloropyridine 2 into the diazotization step without recourse to recrystallization and filtration.

The selective chlorination method described above can be carried out in the presence of about 3 to about 20, about 5 to about 15, mol equivalents of concentrated aqueous hydrochloric acid to 3-aminopyridine 3 and about 0.6 to about 1.5, about 0.8 to about 1.2 mol equivalents of hydrogen peroxide or chlorine to 3-aminopyridine 3. The concentration of the hydrochloric acid can range from about 30 to about 37 wt %. In one embodiment a maximum HCl concentration is used in order to obtain an optimum reaction rate and selectivity in the chlorination step. The chlorination can be accomplished by adding about 30 to about 50 wt % aqueous hydrogen peroxide at a temperature ranging from about 0 to about 60° C. over 1 to 8 hours to a mixture of 3-aminopyridine 3 and the concentrated hydrochloric acid. Alternatively, chlorination can be accomplished by adding chlorine gas at a temperature ranging from about 0 to about 35° C. until >90% conversion of 3-aminopyridine 3. In one embodiment the chlorination temperature ranges from about 10 to about 35° C. for reasons of selectivity and reaction rate. A reaction yield of about 70 to about 80% can be obtained at >90% conversion of 3-aminopyridine 3.

In order to isolate the crude solution of 3-amino-2-chloropyridine hydrochloric acid salt from the mixture, the overchlorinated by-products can be removed by the modified Ieno method, i.e., selective extraction of the byproducts with a non-water-miscible organic solvent such as diethyl ether, ethyl acetate, toluene, benzene or chlorobutane after partial neutralization of the reaction mixture to a pH of about 0.3 to about 1.0 with an inorganic base such as sodium hydroxide, potassium hydroxide, or sodium carbonate. The 3-amino-2-chloropyridine 2 remaining in the aqueous solution can then be extracted with the same organic solvent or another suitable organic solvent after further neutralization of the aqueous solution to a pH of about 2 to about 8. This procedure can leave most of the unconverted 3-aminopyridine 3 in the aqueous waste. The organic extract containing the 3-amino-2-chloropyridine 2 can be extracted with aqueous hydrochloric acid and the aqueous extract can be subsequently used in the diazotization reaction as described above. Alternatively, the organic extract can be concentrated and the resulting crude 3-amino-2-chloropyridine 2 can be further processed to 2,3-dichloropyridine 1 as described above.

As shown in Scheme 3, one embodiment of the present invention relates to an efficient and concatenated process to prepare 2,3-dichloropyridine 1 without having to isolate intermediate solids, e.g., Method C or Method C′. The process involves Hofmann rearrangement of nicotinamide 4 to form 3-aminopyridine 3, selective chlorination of 3-aminopyridine 3 with a suitable chlorinating agent, such as described above in Method B or Method B′, diazotization of the 2-chloro-3-aminopyridine 2, and decomposition of the diazonium chloride salt with copper catalyst wherein at least about 50% of the copper is the copper(II) oxidation state, such as described above in Method A.

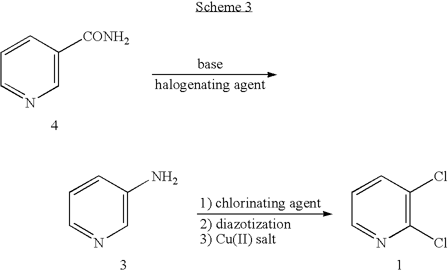

Nicotinamide 4 is a readily available and cost effective precursor to prepare 3-amino-2-chloropyridine 2 and/or 2,3-dichloropyridine 1. Hofmann rearrangement of nicotinamide 4 to form 3-aminopyridine 3 can be achieved in the presence of a suitable halogenating agent and a strong base. The suitable halogenating agent can be, for example but not limitation, chlorine, bromine, hypochlorous acid, hypobromous acid, alkali metal (such as lithium, sodium or potassium) hypochlorite, alkali metal hypobromite, or benzyltrimethyl ammonium tribromide. In one embodiment, a halogenating agent of the present invention is chlorine, bromine, or sodium hypochlorite. A suitable strong base can be an alkali metal hydroxide including but not limited to sodium hydroxide, i.e. caustic. For Hofmann rearrangement references see Org. Synthesis, 1950, 30, 3; U.S. Pat. No. 4,082,749; Chemistry Letters, 1989, 3, 463. Y. Ahmad and D. H. Hey (J. Chem. Soc., 1954, 4516) have described a procedure to convert nicotinamide 4 to 3-amino-2-chloropyridine 2 without having to isolate the 3-aminopyridine 3 intermediate.

In one embodiment of the process of the present invention, a modified Hofmann rearrangement is used involving N-halonicotinamide salt formed under feed-controlled conditions, wherein the molar equivalent of strong base used relative to nicotinamide 4 may be higher than that typically employed in such rearrangements. The modified Hofmann rearrangement can be carried out by co-feeding about 0.8 to about 2.0 equivalents of about 5 to about 15 wt % halogenating agent in an aqueous solution and about 1.0 to about 5.0 equivalents of about 10 to about 50% aqueous strong base to a 10 to 30 wt % nicotinamide aqueous mixture at a temperature ranging from about −5 and about 20° C. and maintaining the pH of the reaction mixture higher than about 10. In one embodiment the temperature ranges from about 0 to about 10° C. The resulting solution of N-halonicotinamide salt is then added to about 1 to about 10 volumes of water in a second reactor over about 0.5 to about 3 hours and the resulting aqueous mixture is maintained at a temperature ranging from about 65 to about 100° C. In one embodiment the reaction temperature is about 70 to about 95° C. for reason of reaction rate. In another embodiment about 3 to about 4 equivalents of strong base to nicotinamide 4 is used to minimize the formation of the byproduct di(3-pyridyl)urea when the halogenating agent is chlorine or bromine. In yet another embodiment about 1 to about 2 equivalents of strong base to nicotinarnide 4 is used when the halogenating agent is sodium hypochlorite. In a further embodiment about 0.9 to about 1.1 equivalents of halogenating agent to nicotinamide 4 is used. The modified Hofmann rearrangement can provide a very high reaction yield. The resulting mixture, comprising crude 3-aminopyridine 3, can be carried onto the chlorination step as described above in Method B or Method B′ after acidification with an acid to a pH of about 1 to about 5. To obtain an optimum rate and selectivity in the chlorination of 3-aminopyridine 3, which requires maximum HCl concentration, the acidified mixture can be concentrated to about 10 to about 30 wt % 3-aminopyridine 3 and then added to about 7 to about 15 equivalents of gaseous HCl. In one embodiment, the 3-aminopyridine 3 can be isolated from the resulting aqueous mixture by extracting with organic solvents and concentration of the organic extracts to afford crude 3-aminopyridine 3, then further purified by crystallization. The isolated 3-amninopyridine 3 can be used in the chlorination step as described above in Method B or Method B′. It is believed that one skilled in the art using the preceding description can utilize the present invention to its fullest extent. The following Examples are, therefore, to be construed as merely illustrative, and not limiting of the disclosure in any way whatsoever. Percentages are by weight except for where otherwise indicated. Quantitative HPLC of the product was performed using a Zorbax Eclipse XDB-C8® pre-packed chromatography column (reversed phase column manufactured by Agilent Technologies, Palo Alto, Calif. 94303) (3 μm particle size, 4.6 mm×15 cm, eluent 15-95% acetonitrile/0.05% TFA/water).

### EXAMPLE 1

### Preparation of 2,3-dichloropyridine 1

To a 300-mL sidearm flask was charged 12.8 g (0.10 mmol) of commercial 3-amino-2-chloropyridine 2, 30 mL of water, and 30 mL of 37% aqueous HCl. After the mixture was cooled to −8° C. (a slurry forms), a solution of 7.0 g (0.10 mol) of NaNO₂ in 14 mL of water was added over 30 minutes at −7 to −3° C. The orange solution became a thin yellow suspension towards the halfway-point of the addition. After the addition, the mixture including the diazonium chloride salt was transferred to a jacketed addition funnel at 0° C. The diazonium chloride salt mixture was added dropwise to a flask containing 20 mL of 37 % aqueous HCl, 60 mL of n-BuCl, and 4.5 g of CuO at 55-62° C. under nitrogen.

The reaction mass was diluted with 100 mL of water and the n-BuCl layer was separated, washed with water, and concentrated to dryness to yield 13.8 g crude 2,3-dichloropyridine 1 as a pale yellow solid (92% yield) with 98% purity.

### EXAMPLE 2

### Preparation of 3-amino-2-chloropyridine 2 using hydrogen peroxide

3-Aminopyridine 3 (30.0 g, 0.32 mole) was add to 300 mL of 37% aqueous HCl in a 1-L Morton flask with overhead stirring at about 30-35° C. After the mixture was cooled to about 10° C., 23 g (0.34 mol) of 50% hydrogen peroxide was added over 20 minutes at about 10-12° C. The mixture was held at about 10° C. for 2 hours and then was allowed to warm to about 19° C. over 2 hours and held at that temperature for additional 4 hours. HPLC analysis showed approximately 90% conversion of 3-amninopyridine 3. After cooling the reaction mixture to 10° C., a solution of 6 g of sodium sulfite in 50 mL of water was added. To the mixture were added 50 mL of toluene and 200 g (2.5 mol) of 50% aqueous sodium hydroxide at about 25-35° C. Then water was added to dissolve precipitated NaCl, and the layers were separated. The organic phase was back-extracted with 45 g of 10% aqueous HCl to recover some 3-amino-2-chloropyridine 2 in the toluene extract, and this was added back to the original aqueous phase. The combined aqueous phases were neutralized to pH 3 with 50% aqueous NaOH and extracted with toluene for 3 times. The toluene extracts were combined, washed with 30 mL of saturated aqueous NaCl, and concentrated to dryness to afford 33 g of crude 3-amino-2-chloropyridine 2 (76% yield) with 94% purity. The product contained about 3 wt % 3-amino-2,6-dichloropyridine by HPLC assay.

### EXAMPLE 3

### Preparation of 3-amino-2-chloropyridine 2 using chlorine

3-Amninopyridine 3 (21.0 g, 0.223 mol) was added to 90 mL (ca. 108 g, 1.08 mol) of concentrated aqueous HCl (ca. 37%) in a 300-mL sidearm flask with magnetic stirring at 30-35° C. The mixture was cooled to 15° C. (thick slurry) and chlorine gas was sparged just above the surface over about 1.5 hours at 15-20° C. HPLC analysis showed approximately 93% conversion of 3-aminopyridine 3. The mixture was cooled to 10° C. and a solution of 6 g of sodium sulfite in 50 mL of water was added. To the mixture was added 30 mL of toluene and 80 g (1.0 mol) of 50% aqueous sodium hydroxide at about 25-40° C. Then water was added to dissolve precipitated NaCl, and the layers were separated. The aqueous phase was extracted once more with 30 mL of toluene. To the aqueous phase was added 10 g of 50% NaOH, and extracted with another 50 mL of toluene to remove 3-amino-2,6-dichloropyridine. The combined organic phase was back-extracted with 40 mL of 0.2 N aqueous HCl to recover some 3-amino-2-chloropyridine 2 in the toluene extracts, and this was added back to the original aqueous phase. The combined aqueous phases were diluted with 100 mL of toluene and neutralized to pH 3 with about 20 g of 50% aqueous NaOH at about 35° C. The aqueous phase was extracted with two 50-mL portions of toluene. The toluene layers were combined and washed with 20 mL of saturated aqueous NaCl. The solution was concentrated to dryness to afford 21.4 g of crude 3-amino-2-chloropyridine 2 (74% yield) with 98.6% purity, which contained about 1.4 wt % 3-amino-2,6-dichloropyridine.

### EXAMPLE 4

### Preparation of 3-amino-2-chloropyridine 2 from nicotinamide 4

To a 200-mL sidearm flask were charged 12.2 g (0.100 mol) of nicotinamide 4 and 60 mL of water and the mixture was cooled to about 5° C. Sodium hypochlorite (63 g, 11.8 wt % aqueous solution, 0.100 mol) was added to the mixture over 30 minutes at 0-5° C. along with 14 g (0.175 mol) of 50% aqueous NaOH over 30 minutes at 0-5° C. to form an N-chloronicotinamide solution. Meanwhile, a second flask (500-mL) was charged with 80 mL of water, which was heated to 80° C. The N-chloronicotinamide solution from the first flask was then transferred to the second flask over 40 minutes, maintaining the reaction temperature at about 75-81° C. The residue in the first flask was rinsed with 20 mL of water and the residual was also transferred to the second flask. The resulting solution was maintained at 80° C. for 15 minutes after the transfer was complete and then was cooled to 40° C. Concentrated aqueous HCl (30 g, 37%, 0.30 mol) was added carefully at 40-50° C. to the solution and the mixture was concentrated at a reduced pressure (ca. 50 mm Hg) until about 160 mL of water was collected. The mixture was cooled to 15° C. and anhydrous HCl (35.2 g, ca.1 mol) was added at 15 to 20° C. The mixture was further cooled to 10° C. and 10.5 g (ca. 0.11 mol) of 32% aqueous H₂O₂ was added over 1.5 hours. After 2 hours at ambient temperature, additional 1 g of H₂O₂ was added and the mixture was held for an additional 30 minutes (ca. 93% conversion). To the mixture was added sodium bisulfite (10 mL, 30% aqueous solution), 100 mL of water, 30 mL of toluene, and 67 g of 50% aqueous NaOH sequentially at 15-25° C. The toluene layer was separated, and the aqueous layer was washed with 30 mL of toluene. The aqueous layer was basified with 4 g of 50% aqueous NaOH to pH 3 and the product was partially extracted with toluene and then with dichloromethane. Additional product was extracted from the aqueous phase after basification to pH 7. The combined organic extracts were concentrated. The residue was dissolved in dichloromethane, and the resulting solution was washed with aqueous NaCl and concentrated to dryness to afford 10.4 g of 3-amino-2-chloropyridine 2 (74% overall yield) with 95% purity.

### EXAMPLE 5

### Preparation of 2,3-dichloropyridine 1 from nicotinamide 4

To a mixture of 24.4 g (0.200 mol) of nicotinamide 4 and 120 mL of water at about 0° C. was added sodium hypochlorite (237 g, 6.89 wt % aqueous solution, 0.22 mol) over 30 minutes. After stirring for over 15 minutes at 0° C., aqueous NaOH (32 g, 0.40 mol, 50 wt %) was added to the mixture over 30 minutes at 0-5° C. This resulting solution was charged to 280 mL of water at 90° C. over 30 minutes and stirred an additional hour at 90° C. Concentrated aqueous HCl (60 g, 37 wt %, 0.20 mol) was added over 45 minutes at 40° C. and the mixture was stirred overnight and concentrated at reduced pressure to remove most of the water. The mixture was then filtered to remove salt, which was washed with two 80 mL portions of 9% aqueous HCl. Analysis of the filtrate showed that it contained about 16.1 g of 3-aminopyridine 3 (ca. 86% yield). To the crude 3-aminopyridine 3 solution was added anhydrous HCl (ca. 80 g, 2.2 mol) at 0° C. Hydrogen peroxide (17.6 g, 46% solution, 0.24 mol) was added over 2 hours at 0-5° C., and the mixture was stirred at 15-20° C. for an additional 3 hours. To the mixture was added aqueous sodium bisulfite solution (12 mL, 30%), water (200 mL), toluene (50 mL), and aqueous NaOH (82 g, 1.03 mol, 50%) sequentially at about 0-20° C. The layers were separated. The aqueous layer was washed with ten 50 mL portions of toluene to remove overchlorinated byproducts, and then basified to pH 10 with 20 g of 50% aqueous NaOH. The basified aqueous solution was extracted with four 100 mL portions of toluene and the combined toluene extracts were washed with two 40 mL portions of 18 wt % aqueous HCl. HPLC analysis of the resulting aqueous HCl extracts showed it contained about 15.3 g (0.119 mol) of 3-amino-2-chloropyridine 2 (ca. 69.7% yield from 3-aminopyridine 3, 60% from nicotinamide 4). These extracts were cooled to about −5° C. and a solution of 8.3 g of sodium nitrite (0.12 mol) in 16.6 mL of water was added over 30 minutes at about −5 to 0° C. The resulting mixture was charged over 1 hour to a mixture containing cupric chloride dehydrate (10.14 g, 0.0595 mol), concentrated aqueous HCl (24.3 mL) and 1-chlorobutane (72 mL) at about 60° C. under a nitrogen atmosphere. After an additional 30 minutes at 60° C., the mixture was cooled to ambient temperature and diluted with 120 mL of water. The layers were separated. The aqueous layer was extracted with two 70 mL portions of 1-chlorobutane. The combined extracts were found to contain about 14.7 g of 2,3-dichloropyridine 1 (83.6% yield from 3-amino-2-chloropyridine 2, or 50% from nicotinamide 4).

What is claimed is:

1. A method for preparing 2,3-dichloropyridine 1, 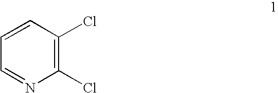
comprising the steps of:
(1) contacting a 3-amino-2-chloropyridine 2 or a solution comprising 3-amino-2-chloropyridine 2 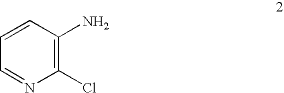
with hydrochloric acid to form a 3-amino-2-chloropyridine hydrochloric acid salt;
(2) contacting the 3-amino-2-chloropyridine hydrochloric acid salt with a nitrite salt to form a corresponding diazonium chloride salt; and
(3) contacting the corresponding diazonium chloride salt with hydrochloric acid in the presence of a copper catalyst wherein at least about 50% of the copper is the copper(II) oxidation state, optionally in the presence of an organic solvent, to form 2,3-dichloropyridine 1.

2. The method of claim 1 wherein the nitrite salt is sodium nitrite.

3. The method of claim 1 wherein at least about 75% of the copper is the copper(II) oxidation state.

4. The method of claim 3 wherein at least about 90% of the copper is the copper(II) oxidation state.

5. The method of claim 4 wherein at least about 95% of the copper is the copper(II) oxidation state.

6. The method of claim 5 wherein at least about 99% of the copper is the copper(II) oxidation state.

7. The method of claim 6 wherein 100% of the copper is the copper(II) oxidation state.

8. The method of claim 1 wherein the copper catalyst comprises copper(II) chloride or copper(II) oxide.

9. The method of claim 8 wherein the nominal mole ratio of the nitrite salt to the 3-amino-2-chloropyridine 2 is about 0.95 to about 2.0; the nominal mole ratio of the copper(II) chloride or copper(II) oxide to the 3-amino-2-chloropyridine 2 is about 0.05 to about 2.0 when 100% of the copper is copper(II) chloride or copper(II) oxide; the nominal mole ratio of hydrochloric acid to the 3-amino-2-chloropyridine 2 in step (1) is about 3 to about 10; and the nominal mole ratio of hydrochloric acid to the 3-amino-2-chloropyridine 2 in step (3) is about 0 to about 10.

10. The method of claim 9 wherein the nominal mole ratio of the nitrite salt to the 3-amino-2-chloropyridine 2 is about 0.95 to about 1.1; the nominal mole ratio of the copper in the copper catalyst to the 3-amino-2-chloropyridine 2 is about 0.2 to about 0.6; the nominal mole ratio of the hydrochloric acid to 3-amino-2-chloropyridine 2 in step (1) is about 3 to about 6; and the nominal mole ratio of the hydrochloric acid to the 3-amino-2-chloropyridine 2 in step (3) is about 1 to about 5.

11. The method of claim 1 wherein steps (1) and (2) are conducted at a temperature ranging from about −15 to about 20° C.; and step (3) is conducted at a temperature ranging from about 30 to about 90° C.

12. The method of claim 11 wherein steps (1) and (2) are conducted at a temperature ranging from about −10 to about 10° C.; and step (3) is conducted at a temperature ranging from about 50 to about 80° C.

13. The method of claim 1 wherein the 3-amino-2-chloropyridine 2 or the solution comprising the 3-amino-2-chloropyridine 2 is prepared by a method comprising the steps of:
(a) contacting 3-aminopyridine 3 or a solution comprising 3-aminopyridine 3 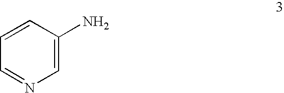
with hydrochloric acid to form a 3-aminopyridine hydrochloric acid salt;
(b) contacting the 3-aminopyridine hydrochloric acid salt with a chlorinating agent to form the solution comprising the 3-amino-2-chloropyridine 2; and
(c) optionally isolating the 3-amino-2-chloropyridine 2 from the solution of step (b).

14. The method of claim 13 wherein the chlorinating agent is chlorine, an alkali metal hypochlorite or a mixture of hydrochloric acid and hydrogen peroxide.

15. The method of claim 14 wherein the chlorinating agent is chlorine or a mixture of hydrochloric acid and hydrogen peroxide.

16. The method of claim 13 wherein the nominal mole ratio of hydrochloric acid to the 3-aminopyridine 3 in step (a) is about 3 to about 20; and the nominal mole ratio of the chlorinating agent to the 3-aminopyridine 3 is about 0.6 to about 1.5.

17. The method of claim 16 wherein the nominal mole ratio of hydrochloric acid to the 3-aminopyridine 3 in step (a) is about 5 to about 15; and the nominal mole ratio of the chlorinating agent to the 3-aminopyridine 3 in step (a) is about 0.8 to about 1.2.

18. The method of claim 13 wherein steps (a) and (b) are conducted at a temperature ranging from about 0 to about 60° C.

19. The method of claim 18 wherein steps (a) and (b) are conducted at a temperature ranging from about 10 to about 35° C.

20. The method of claim 13 wherein the 3-aminopyridine 3 or the solution comprising the 3-aminopyridine 3 is prepared by a method comprising the steps of:
(i) contacting nicotinamide 4 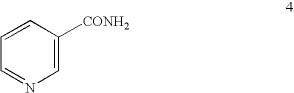
with a strong base and a halogenating agent to form a mixture comprising an N-halonicotinamide salt;
(ii) contacting the N-halonicotinamide salt mixture formed in step (i) with heated water to form an aqueous mixture and maintaining the aqueous mixture at a temperature ranging from about 65 to about 100° C. to form the solution comprising 3-aminopyridine 3;
(iii) isolating the 3-aminopyridine 3 from the solution of step (ii) if the halogenating agent is other than a chlorinating agent; and
(iv) optionally isolating the 3-aminopyridine 3 from the solution of step (ii) if the halogenating agent is a chlorinating agent.

21. The method of claim 20 wherein the strong base is an alkali metal hydroxide.

22. The method of claim 21 wherein the alkali metal hydroxide is sodium hydroxide.

23. The method of claim 20 wherein the halogenating agent is chlorine, bromine, or sodium hypochlorite.

24. The method of claim 20 wherein the nominal mole ratio of the strong base to nicotinamide 4 is about 1 to about 5; and the nominal mole ratio of the halogenating agent to nicotinamide 4 is about 0.8 to about 2.0.

25. The method of claim 24 wherein the nominal mole ratio of the strong base to nicotinamide 4 is about 2 to about 4 when the halogenating agent is chlorine or bromine; the nominal mole ratio of the strong base to nicotinamide 4 is about 1 to about 2 when the halogenating agent is sodium hypochlorite; and the nominal mole ratio of halogenating agent to nicotinamide is about 0.9 to about 1.1.

26. The method of claim 20 wherein step (i) is conducted at a temperature ranging from about −5 to about 20° C.

27. The method of claim 26 wherein step (i) is conducted at a temperature ranging from about 0 to about 10° C.; and step (ii) is conducted at a temperature ranging from about 70 to about 95° C.
